(19) United States
(12) Patent Application Publication
(10) Pub. No.: US 2004/0172035 A1
(43) Pub. Date: Sep. 2, 2004

(54) Tool for expanding the hole formed in the bone, in particular for installing an endosseous dental implant

(75) Inventor: Corrado Saverio Parmigiani, Correggio (IT)

Correspondence Address: BROWDY AND NEIMARK, P.L.L.C., 624 NINTH STREET, NW, SUITE 300, WASHINGTON, DC, 20001-5303, US

(73) Assignee: C.G.M. S.P.A., CORREGGIO (IT)

(21) Appl. No.: 10/778,044

(22) Filed: Feb. 17, 2004

(30) Foreign Application Priority Data
Feb. 27, 2003 (IT) RE2003A000024

(51) Int. Cl. A61B 17/16

(52) U.S. Cl. 606/80

(57) ABSTRACT

The expansion tool comprises an expander body (10) having, for penetration into the hole, an at least partly tapered elongate rectilinear penetration portion of circular cross-section provided at least partly with a helical thread (12) for engaging the wall of the hole; the expansion tool is intended to be engaged by a drive means able to rotate it with torsion about its axis (A), and is characterized in that said expander body (10) is constructed of synthetic resin and is traversed by a rigid axial core (13) positioned in its interior along its axis, and to which the body (10) is bonded.

An important advantage is that as the expander body is of synthetic resin, its manufacturing cost is relatively low to the point that the expander body can be disposed of after use. This means that the use of the tool is very hygienic and at the same time the need for laborious tool cleaning operations is eliminated.

### FIELD OF THE INVENTION

The present invention relates in particular to the installation of endosseous dental implants.

The installation of endosseous dental implants is known to require the formation of holes in the mandible or in the maxilla, into which the threaded shank of the implant is inserted.

In an advanced method, a hole of substantially smaller diameter than the shank of the implant is firstly made in the maxilla or mandible by a milling cutter; this hole is then enlarged by inserting into it, with movement in the axial direction, a pointed expansion tool (punch) of greater diameter than the hole, so causing the hole to expand radially; the expansion tool is then extracted and the shank of the desired dental implant is inserted into the hole before the hole in the bone returns to its initial diameter. This method has many advantages:

in particular, it prevents weakening of the bone, especially the maxillary bone, by holes of relatively large diameter, even if the dental implant possesses a endosseous shank of relatively large diameter;

the quality of the implant bone is improved, because of bone condensation;

the dental implant is made stable, even in cases of poor bone quality;

there is a relatively small loss of bone substance.

This method has however certain drawbacks, in particular the fact that the expansion tool is inserted by blows (with a surgical hammer) and hence produces traumatic discomfort in the patient; because of its nature, this procedure is imprecise and requires considerable attention and expertise by the operator.

A more advanced tool, able to overcome the aforesaid drawbacks, is described in U.S. Pat. No. 6,146,138. The tool described therein comprises an expander body having, for penetration into the hole, a tapered rectilinear elongate penetration portion of circular cross-section provided with a helical thread for engaging the wall of the hole. The rear shank of the tool is intended to be engaged in torsional rotation by a usual micromotor drive means for dentistry.

In use, after the apical part of the tool has been placed in the mouth of the hole formed in the bone, the tool is rotated slowly (15-25 r.p.m.) by a usual manual implement or by said motorized means; by engagement against the bone wall of the hole, the helical thread produces a helical kinematic torque between the tool and the hole; consequently the tool rotation also produces axial penetration of the tool along the hole, with consequent widening of the hole diameter because of the larger diameter of that tool part following the apical part, until the hole expansion reaches an extent suitable for inserting the implant.

The axial advancement produced by rotating the tool is very precise and regular, being a function of the number of revolutions undergone; moreover the movement is continuous and relatively smooth, reducing to a minimum any traumatic discomfort to the patient. If a considerable widening of the hole diameter is required, one or more tools having different maximum diameters are inserted into the hole, these being inserted one at a time in succession into the same hole, starting with that of smallest maximum diameter and progressing through those of increasing maximum diameter.

### SUMMARY OF THE INVENTION

An object of the present invention is to improve said expansion tool such as to make its manufacture more economical to the point at which it is convenient to dispose of it after use, hence making its use more comfortable and more hygienic.

This and other objects are attained by the invention as characterized in the claims.

The invention is based on the fact that said expander body is constructed of synthetic resin and is traversed by a rigid axial core positioned in its interior along its axis, and to which the body is bonded.

### BRIEF DESCRIPTION OF THE DRAWINGS

FIG. 1 is a perspective exploded view of the tool of the invention.

FIG. 2 is an axial section through the tool.

FIG. 3A is a side view of the expander body.

FIG. 3B is an axial section through FIG. 3A.

FIG. 3C is a view in the direction of the arrow C of FIG. 3A.

FIG. 3D is a section on the plane D-D of FIG. 3A.

FIG. 4A is an axial section through the rear shank.

FIG. 4B is a view in the direction of the arrow B of FIG. 4A.

FIG. 5A is an axial section through the axial locking element.

FIG. 5B is a section on the plane B-B of FIG. 5A.

FIG. 5C is a section on the plane C-C of FIG. 5A.

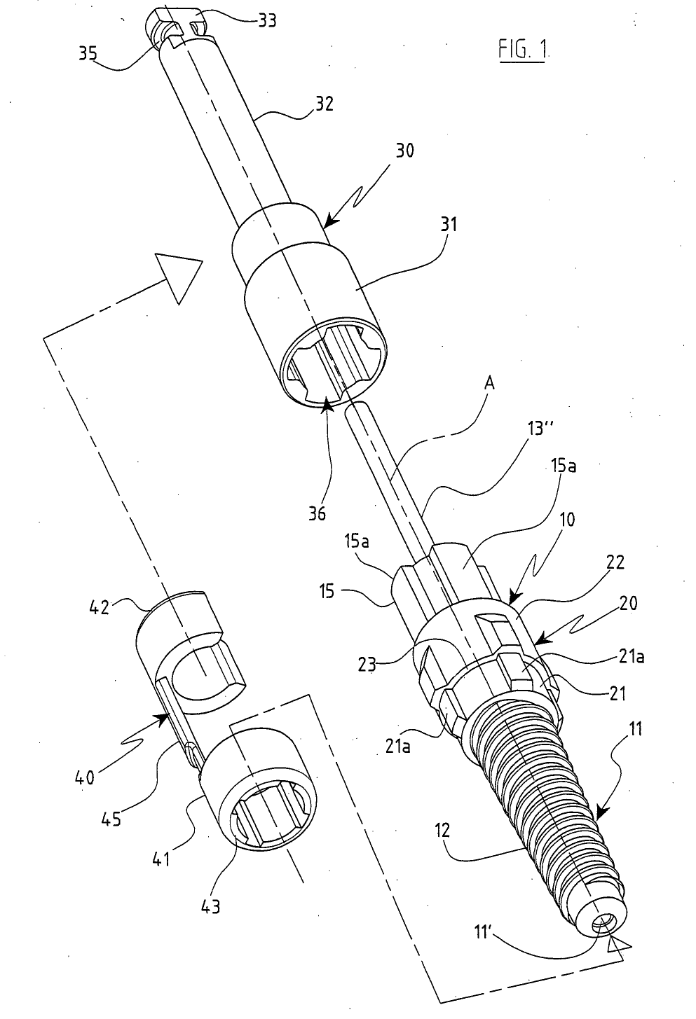

### DESCRIPTION OF THE PREFERRED EMBODIMENT

The illustrated expansion tool is formed substantially of three independent components to be fitted together, namely: an expander body 10, a rear shank 30 and an axial connection element 40.

The expander body 10 comprises, at its front end, to penetrate into the hole 8 provided in the bone 9, an at least partly tapered elongate rectilinear penetration portion 11 of circular cross-section provided at least partly with a helical thread 12 for engaging the wall of the hole 8. In the illustrated embodiment, the entire penetration portion 11 is tapered (specifically frusto-conical) and is totally provided with the thread 12. According to the invention, the expander body is constructed of synthetic resin and is traversed by a rigid axial core 13 positioned in its interior along its axis, and to which the body 10 is bonded.

Preferably, the plastic polymer used is an acetal resin (POM) which is injection molded in a mold by which the core 13 is clad to form a one-piece body with the core (this type of process is known as “plastic overmolding of metal inserts”).

The core 13 is in the form of a thin tube, of which the front end reaches a mouth 11′ located at the front end of the penetration portion 11, and the rear end opens externally at the rear end of the expander body 10, to project outwards from it by a portion 13″.

The rear shank 30 is of metal, its front end portion 31 being arranged to torsionally engage the rear end portion 15 of the expander body 10, so that its axis coincides with the axis of the expander body 10.

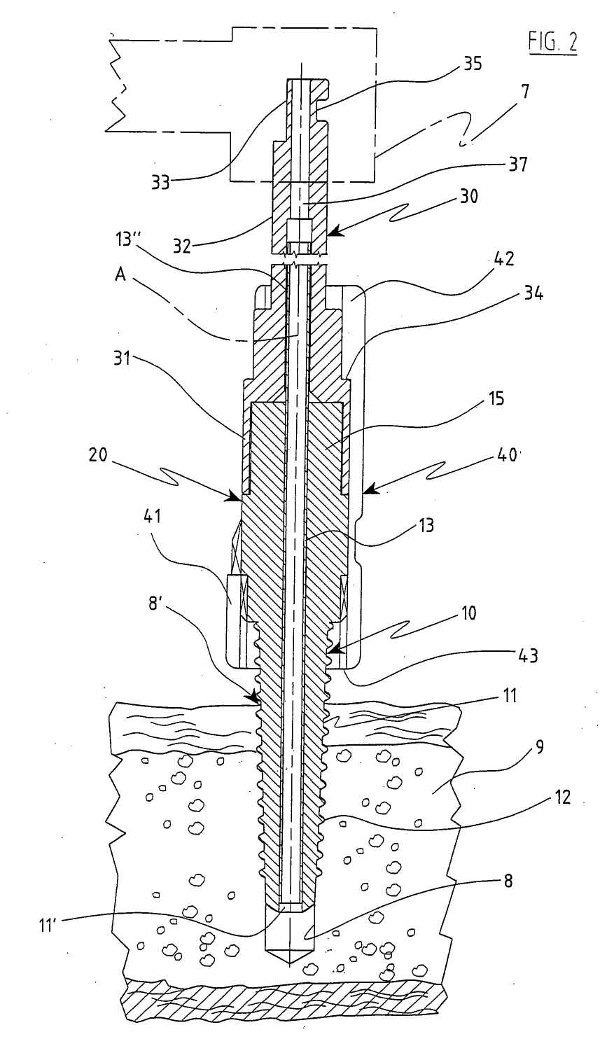

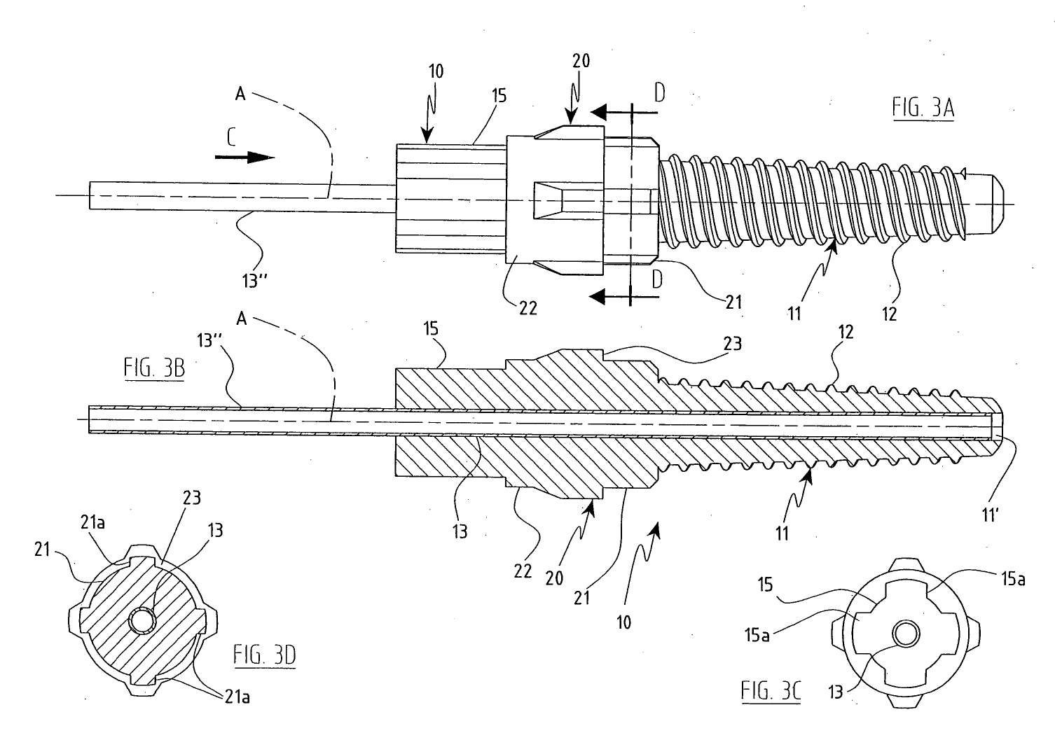

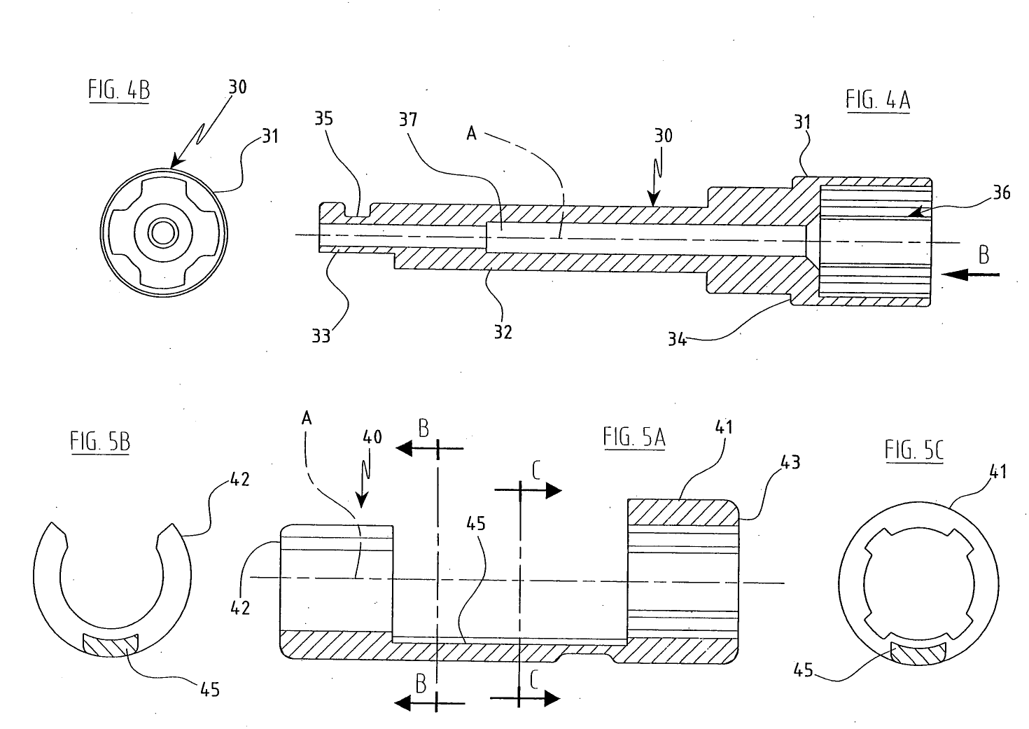

The rear end portion 15 possesses a lateral surface provided with longitudinal grooves which give rise to teeth 15a and corresponding radial recesses; the portion 31 of the shank 30 is in the form of a socket possessing a front-opening cavity 36, the lateral inner surface of which has a shape complementary to the lateral surface of the portion 15 of the expander body 10, to enable prismatic engagement therewith. The rear portion 32 of the shank 30 is in the form of an axial cylindrical stem and is traversed by a through axial cavity 31, the front portion of which has dimensions such as to exactly house the projecting rear portion 13″ of the core 13. The rear portion 32 can be engaged in torsional rotation by a usual drive means for rotating a tool, for example a usual micromotor drive means 7 (of known type shown schematically in FIG. 2). In detail, the shank 30 possesses at its rear end an annular groove 35 and a flat milled face 33, by which it is engaged by the chuck of the micromotor drive means 7 and rotated about the axis A of the tool.

Consequently, by means of the rear shank 30, the expander body 10 can be engaged by the drive means 7 which rotates it with torsion about its axis A.

The drive means 7 is advantageously of the type able to feed cooling/lubricant fluid to the end of the tool, this liquid being fed to the front end of the expansion tool through the axial cavity 37 and the through cavity of the core 13.

The element 40 axially locks the expander body 10 and the rear shank 30 together.

The expander body 10 possesses an intermediate part 20 formed from a front cylindrical portion 21 which projects radially from the penetration portion 11, and a rear cylindrical portion 22 joined to the part 21. The front cylindrical portion 21 has a diameter less than the rear cylindrical portion 22, between the two there being defined a front facing shoulder 23 located to the rear of the penetration portion 11. On its lateral surface the front cylindrical portion 21 also possesses a number of axially extending teeth 21a distributed at the same angular distance apart. The rear cylindrical portion 22 projects radially from the rear end portion 15, its rear end abutting against the front end of the rear shank 30. This also possesses a rear facing second shoulder 34, provided on the outer surface of the portion 31.

The axial locking element 40 comprises a first ring 41 and a second ring 42 joined together by a longitudinal filament 45. The first ring 41 embraces as an exact fit the expander body 10, in particular its front cylindrical portion 21, in a position adjacent to the first shoulder 23, the second ring embracing as an exact fit the rear shank 30, in particular its front portion 31, in a position adjacent to the said shoulder 34.

The first ring 41 is closed and is applied to the expander body 10 by sliding the penetration portion 11 axially through it. The second ring 42 is open and extends through an angle greater than 180 degrees to be snap mounted about the front portion 31, where it abuts against the shoulder 34, after the first ring has been mounted about the cylindrical portion 21 to abut against the first shoulder 23.

The first ring 41 defines a front facing shoulder 43 which, when the element 40 has been applied to the penetration portion 11, is disposed on the rear end region of the portion 11; this shoulder 43 provides an abutment against the outer wall of the bone at the entry mouth 8′ of the hole 8, to halt the penetration of the expansion tool into the hole 8. Advantageously, two or more axial locking elements 40 for association with the same expansion tool are provided, in which the first ring 41 has a different axial dimension, in order to define that number of tool penetration halting shoulders located in different axial positions relative to the penetration portion 11.

A plurality of expander bodies 10 are also advantageously provided, their penetration portions 11 having different diameters one from the other, to be inserted one at a time in succession into the same bone hole 8, starting with that of smallest maximum diameter and progressing through those of increasing maximum diameter.

In use, after the apical part of the penetration portion 11 has been placed in the initial part of the hole 11, the expansion tool is rotated slowly (15-25 r.p.m.) by means of a usual manual or motorized implement; by engagement against the bone wall of the hole, the helical thread 12 produces a helical kinematic torque between the tool and the hole; consequently the tool rotation also produces axial penetration of the tool along the hole 8, with consequent widening of the hole diameter because of the larger diameter of that part of the penetration portion 11 following the apical part, until the hole expansion reaches an extent suitable for inserting the implant.

If a considerable widening of the hole diameter is required, after a relatively narrow hole has been formed in the bone a small series of expansion tools with different diameters of their penetration portions 11 is used, to be inserted one at a time into the same hole starting with that of smallest maximum diameter, until the achieved expansion of the hole 8 is sufficient for inserting the implant.

The axial advancement produced by rotating the tool is very precise and regular, being a function of the number of revolutions undergone; moreover the movement is continuous and relatively smooth, hence reducing to a minimum any traumatic discomfort to the patient. The extent of tool penetration can be controlled by controlling the angle through which the tool is rotated.

As the expander body 10 is of synthetic resin, its manufacturing cost is relatively low to the point that the expander body can be conveniently disposed of after use. This means that the use of the tool is very hygienic and at the same time the need for laborious tool cleaning operations is eliminated.

As the rear shank 30 does not come into direct contact with the hole formed in the bone, it can advantageously be used an indefinite number of times; it is therefore of metal construction, its cleaning and sterilization hence not posing particular problems.

Another important advantage is the fact that as the expander bodies are for once-only use, different packages can be made up on the basis of the different dimensions (diameter and length) to be given to the endosseous hole, each package containing a number of tools with predetermined increasing diameters, so making it easier for the operator to choose the tool sizes on the basis of the dimensions of the hole to be obtained. Moreover, by using a number of axial locking elements 40 with rings 41 of different axial dimensions, the length of penetration of the tool can be kept under control.

Moreover, as the synthetic resin can be produced in various different colors, expander bodies 10 can be formed of different colors based on a fixed utilization sequence in relation to their different diameters.

Because of the precision and degree of finish with which the synthetic resin bodies can be formed, the penetration portion 11 is free of burrs or other irregular particles which could otherwise damage the bone tissue. Physiological liquid could be injected into the hole 8 through the tubular core 13 during insertion of the expansion tool, so conveniently lubricating and cooling the bone tissue.

Numerous modifications of a practical and applicational nature can be made to the invention, but without leaving the scope of the inventive idea as claimed below.

What is claimed is:

1. An osseous expansion tool for expanding a hole formed in the bone, comprising an expander body (10) having, for penetration into the hole (8), an at least partly tapered elongate rectilinear penetration portion of circular cross-section provided at least partly with a helical thread (12) for engaging the wall of the hole, the expansion tool being engagable by a drive means able to rotate it with torsion about its axis (A), characterized in that said expander body (10) is constructed of synthetic resin and is traversed by a rigid axial core (13) positioned in its interior along its axis, and to which the body (10) is bonded.

2. A tool as claimed in claim 1, characterized in that said axial core (13) is tubular.

3. A tool as claimed in claim 2, characterized in that said axial core (13) has a front end which opens at the front end of the penetration portion (11), and a rear end which opens externally at the rear end (15) of the expander body (10), the tool being engagable by a drive means (7) able to feed cooling/lubricant liquid to the rear end of the axial core (13).

4. A tool as claimed in claim 1, characterized in that said axial core (13) is of metal.

5. A tool as claimed in claim 1, characterized by comprising a rear metal shank (30), the front end portion (11) of which is arranged to be torsionally coupled to the rear end portion (15) of the expander body (10), the rear end portion (32) of the shank (30) being in the form of an axial stem to be engaged in torsional rotation by a usual drive means (7) for rotating a tool.

6. A tool as claimed in claim 5, characterized by comprising an element (40) for axially locking the expander body (10) and the rear shank (30) together.

7. A tool as claimed in claim 6, characterized in that the expander body (10) possesses a front facing first shoulder (23) located to the rear of the penetration portion (11), the rear shank (30) possessing a rear facing second shoulder (34), said axial locking element (40) comprising a first ring (41) arranged to embrace the expander body (10) in a position adhering to the first shoulder (23) and a second ring (42) arranged to embrace the rear shank (30) in a position adhering to the second shoulder (34), said rings being joined together by a longitudinal filament.

8. A tool as claimed in claim 7, characterized in that said first ring (41) defines a front facing shoulder (43) located on the rear region of the penetration portion (11) to provide an abutment against the entry mouth (8′) of the hole (8), to halt the tool penetration into the hole 8.

9. A tool as claimed in claim 8, characterized by comprising at least two axial locking elements (40) for association with the same penetration portion (11), in which the first ring (41) has a different axial dimension in order to define that number of tool penetration halting shoulders (43) located in different axial positions relative to the penetration portion (11).

10. A group of osseous expansion tools, characterized by comprising a plurality of expander bodies (10) claimed in claim 1, their penetration portions (11) having different diameters one from the other, to be inserted one at a time in succession into the same hole (8), starting with that of smallest maximum diameter and progressing through those of increasing maximum diameter.
